(19) United States
(12) Patent Application Publication
(10) Pub. No.: US 2025/0339282 A1
(43) Pub. Date: Nov. 6, 2025

(54) POROUS-BASED TALUS RECONSTRUCTION PROSTHESIS AND ASSOCIATED METHOD

(71) Applicant: OsseoLabs Co., Ltd., Bangkok (TH)

(72) Inventors: Patcharapit Promoppatum, Nonthaburi (TH); Aingfa Pinyonitikasem, Bangkok (TH); Naruporn Jitkla, Bangkok (TH); Jaruat Jampa, Trang Province (TH); Vikram Ahuja, Bangkok (TH)

(21) Appl. No.: 19/195,930

(22) Filed: May 1, 2025

Related U.S. Application Data
(60) Provisional application No. 63/641,691, filed on May 2, 2024.

(51) Int. Cl. A61F 2/42; A61B 17/72; A61F 2/30

(52) U.S. Cl. CPC A61F 2/4202; A61B 17/7291; A61F 2002/30125; A61F 2002/30235; A61F 2002/30242; A61F 2002/30263; A61F 2002/30772; A61F 2002/3092; A61F 2002/30948; A61F 2002/30952; A61F 2002/4207

(57) ABSTRACT

This invention is designed for orthopedic applications. It features a proximal surface for tibial articulation, a distal surface for calcaneal engagement, and a central canal for a fixation nail. Offered in spherical, ellipsoidal, and anatomically contoured shapes, the prosthesis includes a gyroid lattice structure for improved osseointegration. A method for its creation involves 3D modeling from CT scans, shape design, lattice definition, and 3D printing, focusing on dimensions and density variations to ensure proper fit and functionality while addressing anatomical needs.

### CROSS-REFERENCE TO RELATED APPLICATION

The present application claims the benefit of U.S. provisional patent application No. 63/641,691, filed on May 2, 2024. The disclosures of the above-mentioned application are incorporated herein by reference in their entirety.

### FIELD OF THE INVENTION

The present invention relates to a porous-based talus reconstruction prosthesis.

### BACKGROUND OF THE INVENTION

Ankle deformities can come from a variety of causes, including injuries and diseases such as diabetes and cancer. This invention specifically addresses abnormalities in the talus bone, which serves as a connector between the leg and foot. A functional talus enables normal ankle rotation, flexion, and extension. The loss of the talus in ankle deformities can result in restricted mobility and limb shortening as the bones collapse, resulting in a low quality of life for the patient.

For the treatment, the surgeon performs a tibiotalocalcaneal arthrodesis to fuse the ankle joint together. An intramedullary nail (4) is inserted from the heel into the tibia, and screws (6) are used to secure the bone and nail together from the hindfoot and at the side of the calcaneus, preventing further movement.

In cases of high bone volume loss, additional procedures are necessary to address limb shortening. A conventional method is distraction osteogenesis, which involves cutting the leg bone and using a metal cage to support and gradually extend the bone to achieve the desired leg length.

However, this method has drawbacks, including its lengthy duration and the potentially suboptimal strength of bone formation. Alternatively, surgeons may opt to fill the void space with an allograft, such as a femoral head, to reduce the degree of limb shortening. Nevertheless, this procedure is typically feasible only in large hospitals due to the challenge of obtaining the femoral head from donors. Additionally, the surgery becomes more complex as the femoral head needs reshaping and resizing to fit the patient's injury. Moreover, using femoral head allografts increases the risk of infection, especially in patients with diabetes. In severe cases, where limb preservation is not feasible or poses a significant risk to the patient's life, the surgeon may need to consider amputation as a last resort.

The benefit of the porous-based talus prosthesis could be lists as follows.

Reduced Limb Shortening: By reconstructing the talus bone, the prosthesis can help prevent limb shortening that occurs due to bone collapse, thereby improving the patient's limb length and overall posture.

Enhanced Quality of Life: Restoring normal ankle function can significantly improve the patient's quality of life by reducing pain, increasing independence, and enabling participation in daily activities.

Minimized Surgical Complexity: Compared to alternative procedures like distraction osteogenesis or allograft transplantation, the use of a porous-based talus prosthesis may simplify surgical procedures, reducing operative time and potential complications.

Decreased Risk of Infection: Unlike procedures involving allografts, which increase the risk of infection, the use of a prosthesis made from biocompatible materials may lower the risk of postoperative infections, particularly beneficial for patients with conditions like diabetes.

Tailored Fit: The prosthesis can be custom designed with varied radiuses to fit the patient's specific injury, potentially reducing the need for extensive reshaping and resizing during surgery.

Preservation of Natural Anatomy: The porous-based talus prosthesis aims to replicate the structure and function of the natural talus bone, thereby preserving as much of the patient's natural anatomy as possible.

Longevity: A talus prosthesis contains well-defined porous architecture, which could promote the bone integration. Thus, the porous-based device could fuse well with the surrounding bones, offering durability and longevity as well as reducing the need for frequent revision surgeries and promoting long-term functional outcomes for the patient.

### SUMMARY OF THE INVENTION

The present disclosure provides a talus prosthesis designed for orthopedic applications, featuring several key components and configurations. This prosthesis includes a proximal surface for articulation with a tibial component, a distal surface for engagement with the calcaneus, and a body portion with a central cylindrical canal to accommodate a fixation nail. The fixation nail helps secure the prosthesis in place, allowing for stability under physiological loading.

The talus prosthesis comes in three shapes: a spherical design (7) suitable for general replacements, an ellipsoidal shape (8) adaptable to patient-specific needs for severe limb shortening, and an anatomically contoured version (9) that mimics the natural shape of the native talus. The cylindrical canal's diameter varies depending on the intramedullary nail (4) used, ranging from 5 mm to 30 mm.

An internal gyroid lattice structure is incorporated to enhance osseointegration and minimize stiffness mismatch, featuring interconnected pores to support bone growth.

Additionally, a method for creating an ankle prosthesis is outlined, which includes steps like reconstructing a 3D model from CT scans, designing the external shape, determining lattice structures, performing simulations for stress analysis, and utilizing 3D-printing technology. The implants are tailored to account for vertical and horizontal diameters to ensure proper fit and functionality, with variations in density depending on the location and load-bearing requirements.

### BRIEF DESCRIPTION OF THE DRAWINGS

Illustrative embodiments of the present disclosure will be described with reference to the accompanying drawings.

FIG. 1 is an X-ray image of the talus with anomalies.

FIG. 2 is a 3D reconstructed model of the talus with anomalies.

FIG. 3 is an image of a 3D model of a defective talus.

FIG. 4 is a reconstruction method using an intramedullary nail and a porous-based Talus prosthesis for bone reconstruction and enhanced fusion.

FIG. 5 is an example of external shape determination for talus prosthesis, including ellipsoidal with varied radii, spherical, or anatomically conformed talus implant.

FIG. 6 is an Example of porous integration when the external shape of the talus prosthesis was defined by a sphere.

FIG. 7 is a flowchart of the process for creating a Porous-based Talus Reconstruction Prosthesis.

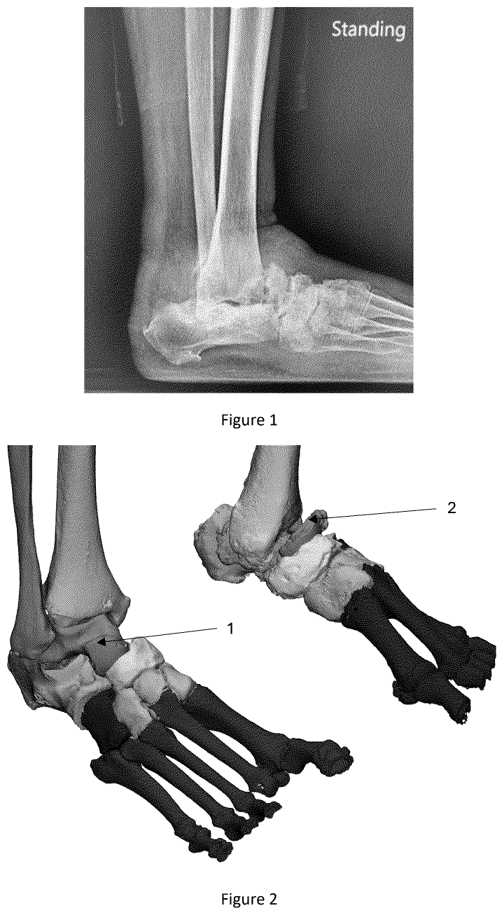

### DETAILED DESCRIPTION OF THE INVENTION

In the following, preferred embodiments of the present invention will be described in detail with reference to the accompanying, exemplary diagrams.

To address the ongoing challenges associated with talus reconstruction in orthopedic surgery, we have engineered an innovative porous-based talus prosthesis (5). This advanced synthetic implant has been meticulously designed for the reconstruction of the talus bone, particularly in cases involving ankle deformities. Its primary goal is to restore normal ankle function by supplying adequate support and stability to the joint, facilitating a return to a functional and pain-free range of motion.

The prosthesis is distinguished by its customizable external dimensions, which allow it to be tailored to accommodate a variety of anatomical shapes and sizes. This adaptability is crucial in ensuring a precise fit for each individual patient. Inside the prosthesis, a network of internal porous structures has been integrated, which may offer several advantages. These include enhanced integration with surrounding bone tissue and a decreased likelihood of postoperative infection. The thoughtful design allows for further customization based on the specifics of the patient's injury, potentially streamlining surgical procedures and improving long-term functional outcomes.

The talus prosthesis consists of several components:

1. A proximal surface designed to articulate seamlessly with a tibial component.

2. A distal surface tailored to engage with the calcaneal surface.

3. A body portion that extends between the proximal and distal surfaces, serving as the main structural element.

4. A cylindrical canal that runs longitudinally through the body from the proximal to the distal surface, specifically engineered to accommodate a fixation nail.

The fixation nail, when inserted through the cylindrical canal, engages neighboring bone structures to create a robust locking mechanism. This secure fit resists any displacement even under the stress of physiological loading, ensuring the prosthesis remains firmly in position during healing and rehabilitation.

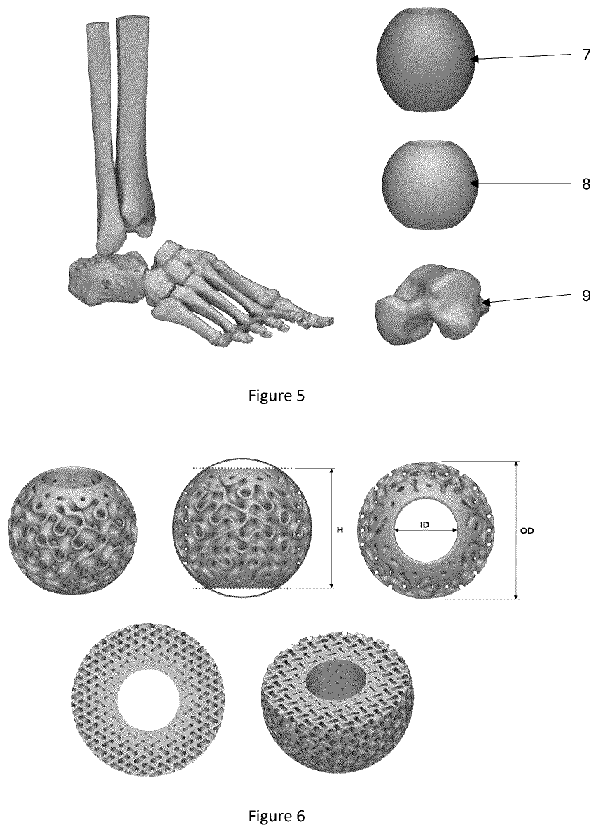

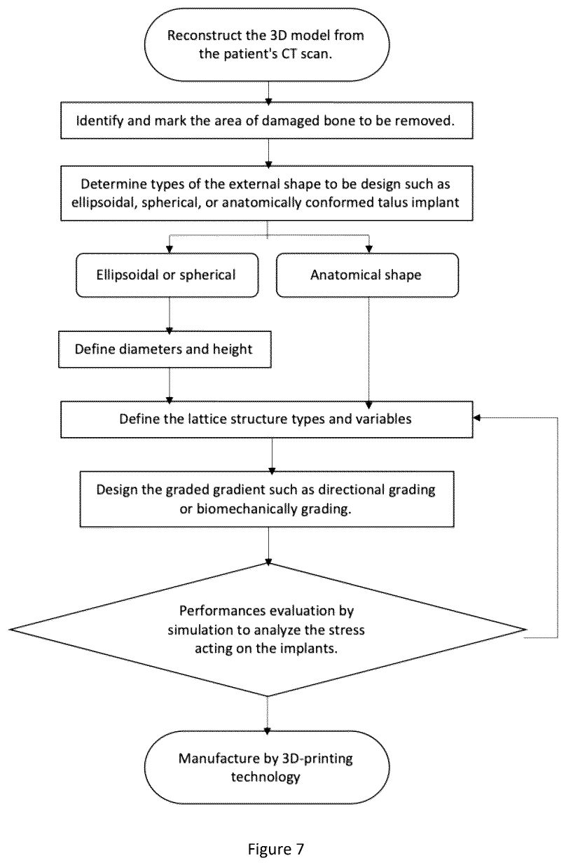

The body portion of our talus prosthesis is offered in three distinct geometric configurations (FIG. 5), each crafted to meet different clinical requirements and anatomical variations:

Spherical Design (7): This configuration features a range of diameters from 10 mm to 60 mm, providing symmetrical articulation and facilitating straightforward alignment. It is particularly well-suited for generalized replacements in the ankle.

Ellipsoidal Shape (8): This variant is defined by its adjustable anterior-posterior and medial-lateral dimensions, allowing for precise adaptation to the unique anatomical needs of patients. This shape is especially beneficial in cases involving severe limb shortening, as the vertical height aids in restoring the limb's original length. The horizontal diameter can vary from 10 mm to 60 mm, while the vertical dimension can extend from 10 mm to 100 mm.

Anatomically Contoured Shape (9): This configuration replicates the natural geometry of the native talus, an asymmetrical bone composed of three primary regions: the body, neck, and head. The body presents a roughly cuboidal shape with a dome-shaped surface that articulates with the tibia, measuring approximately 10-60 mm in width anteriorly and 10-60 mm posteriorly. The curvature radius varies between 10-30 mm depending on individual anatomy. The neck is notably oblique, angling medially at around 10-30 degrees in relation to the body's long axis, and slopes downward and forward. The head, which is convex and directed anteromedially, engages with the navicular bone. Inferiorly, the talus showcases three articular facets—posterior, middle, and anterior—facilitating articulation with the calcaneus and forming the sophisticated structure of the subtalar joint.

The cylindrical canal within the prosthesis can vary from 5 mm to 30 mm in diameter, tailored to suit the size of the intramedullary nail (4) used during tibiotalocalcaneal (TTC) arthrodesis.

Common across all designs is the inclusion of an internal gyroid lattice structure—a type of triply periodic minimal surface (TPMS)—which is critical for fostering osseointegration. This lattice configuration features an interconnection of pores ranging from 300 to 3000 microns, which promotes vascularization and bone ingrowth, all while preserving mechanical strength and the stability of the implant. The surface of the cylindrical canal in contact with the intramedullary nail (4) has a relative density that ranges from 0.1 to 1.0, aiming to match the stiffness of the implant with that of the nail, further optimizing integration.

By combining a modular approach to prosthesis shape, a fixation-enabling cylindrical canal, and a biofunctional porous architecture, our talus prosthesis is designed to restore optimal ankle joint stability while supporting long-term biological integration with surrounding tissues. This innovative solution represents a significant advancement in the field of orthopedic implants, promising improved outcomes for patients with ankle deformities.

### Design Concept

The design process of talus implants comprises two primary steps, which are the external shape determination and the internal structure integration.

### 1. External Shape Determination

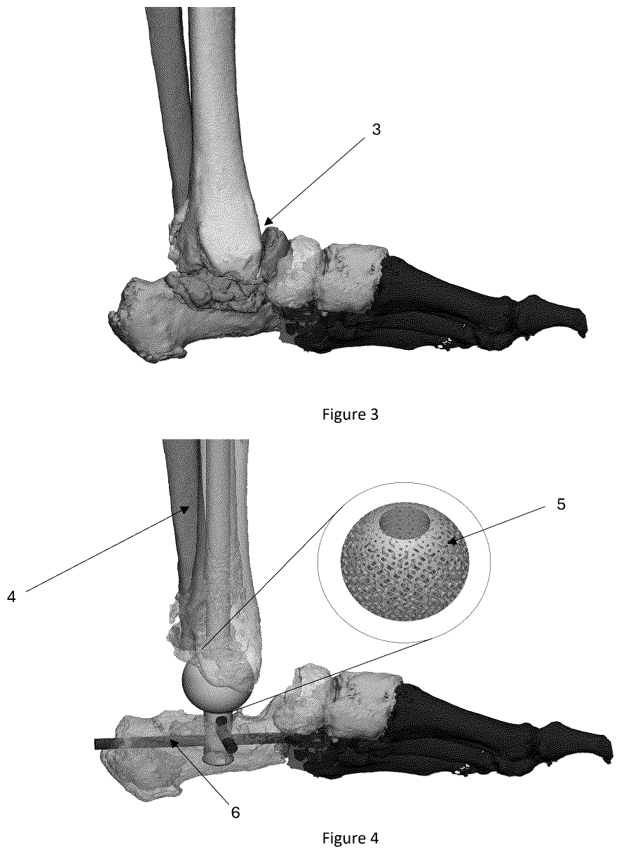

A 3D model of the ankle bones is generated from the patient's CT scan (FIGS. 2 and 3). The extent of the damage is assessed, and a surgical plan is developed to determine the area of bone removal (FIG. 4). Then, the talus implants are designed to fill the volume of the removed bone. The external shape can be outlined as either an ellipsoid with varied radii, spherical, or anatomically conformed talus implants, to use with the intramedullary nail (4), or as an anatomical talus implant intended to replicate the function of the original talus. The ellipsoidal and/or spherical talus implants are designed with two parameters: the vertical diameter and the horizontal diameter. The vertical diameter controls the height of the implants, addressing the challenge of limb shortening. Meanwhile, the horizontal diameter is considered to ensure that the implants fit securely within the tibia bone region and can accommodate the surrounding skin. After the parameters are determined, a cylindrical volume will be subtracted from the implants to allow the intramedullary nail (4) to pass through. On the other hand, anatomical talus implants can be obtained by mirroring the talus from the patient's unaffected ankle.

The vertical diameter (dv) and the horizontal diameter (dh) of ellipsoidal and spherical external shapes can be used to generate the corresponding geometries based on the following equation.

$$\frac{{x}^{2}}{{( \frac{{d}_{h}}{2} )}^{2}} + \frac{{y}^{2}}{{( \frac{{d}_{v}}{2} )}^{2}} = 1$$

Meanwhile, the height of the implants can be calculated using the equation below.

$$\frac{{r}_{c}^{2}}{{( \frac{{d}_{h}}{2} )}^{2}} + \frac{{( \frac{h}{2} )}^{2}}{{( \frac{{d}_{v}}{2} )}^{2}} = 1$$

dₕ is the horizontal diameter of the ellipsoidal and spherical external shapes.
dᵥ is the vertical diameter of the ellipsoidal and spherical external shapes.
r_{c} is the radius of the inner cylinder that passes through the external shape.
h is the height of the implant, which is coaxial with the intramedullary nail.

### 2. Internal Structure Integration

The internal porous structure is designed. The porous size and distribution can be determined by considering the function of the talus implants, such as osseointegration properties, bone contact regions, screw insertion points, and load-bearing performance. For example, the ellipsoidal and spherical talus implants used with an intramedullary nail (4), their primary function is to fill the void space and promote bone growth within the implants. As osseointegration is the main objective, the lattice cell size, pore size, and relative density are designed to facilitate optimal osseointegration, while the load-bearing function is considered secondary. As a result, the porous gradient structure is consistently graded from the inner cylinder in contact with the intramedullary nail (4) to the outer diameter in the horizontal direction. For example, a Triply Periodic Minimal Surface (TPMS) structure can be functionally utilized to manipulate the changes in the graded structure according to the equation by Poltue et al. (Poltue, T., Karuna, C., Khrueaduangkham, S., Seehanam, S., and Promoppatum, P., 2021. Design exploration of 3D-printed triply periodic minimal surface scaffolds for bone implants. International Journal of Mechanical Sciences, 211, p.106762.)

$${ρ}^{*} = t \frac{{m}_{1}}{L}$$

While ρ* is the relative density
t is the effective wall thickness of TPMS structures
m₁ Is the constant for correlations of relative density
L is the unit cell size

$${d}_{p o r e} = {m}_{2} L - t$$

When dₚₒᵣₑ is the TPMS pore size
m₂ is the constant for correlations of pore size

| TPSM Structure | m₁ | m₂ |
|---|---|---|
| Primitive | 2.35 | 0.94 |
| Gyroid | 3.09 | 0.50 |
| Diamond | 3.84 | 0.50 |
| Neovius | 3.63 | 0.83 |
| FRD | 4.86 | 0.57 |
| IWP | 3.55 | 0.57 |

The area in contact with the nail has a higher relative density to account for friction on the surface, while the outer surface has a lower relative density to reduce weight, as it is not subjected to forces. On the contrary, the functions of anatomical talus implants are more complex, as they need to provide osseointegration, load-bearing performance, and facilitate movement of the ankle. The distribution of porosity can be determined through simulations of daily activities, allowing observation of the stress acting on the implants. Porosity can then be distributed according to the relative forces acting on the implants. FIG. 6 shows the example of porous integration when the external shape of the talus prosthesis was defined by the spherical. Additionally, finite element analysis can be incorporate to validate the desire performance of the final implant. Finally, the talus implant can be manufacture by using the 3D-printing technology.

### REFERENCE NUMBER

1 Normal Talus (Right Foot)

2 Collapsed Talus (Left Foot)

3 Collapsed Talus

4 Intramedullary nail for ankle fusion

5 Talus Replacement Sphere

6 Screws

7 Ellipsoidal shape prosthesis

8 Spherical shape prosthesis

9 Anatomical shape prosthesis

What is claimed is:

1. A talus prosthesis, comprising: (i) a proximal surface configured to articulate with a tibial component; (ii) a distal surface configured to engage with a calcaneal surface; (iii) a body portion extending between the proximal and distal surfaces; and (iv) a central cylindrical canal extending longitudinally through the body portion from the proximal surface to the distal surface, with the central canal configured to receive a fixation nail;
wherein the fixation nail, when inserted through the cylindrical canal, engages with adjacent bone structures to establish a locking mechanism that secures the prosthesis in position and resists displacement under physiological loading;
wherein the body portion of the talus prosthesis is available in three geometric configurations, each designed to accommodate different clinical scenarios and anatomical variations.

2. The talus prosthesis of claim 1, wherein a spherical design, with diameters ranging from 10 mm to 60 mm, provides symmetric articulation and simplified alignment, suitable for generalized replacements.

3. The talus prosthesis of claim 1, wherein an ellipsoidal shape that varies in anterior-posterior and medial-lateral dimensions provides flexibility for adapting to patient-specific anatomical needs, making it suitable for cases of severe limb shortening by restoring the limb's original length, with a horizontal diameter ranging from 10 mm to 60 mm and a vertical diameter from 10 mm to 100 mm.

4. The talus prosthesis of claim 1, wherein the cylindrical canal ranges from 5 mm to 30 mm in diameter, depending on the size of the intramedullary nail used for tibiotalocalcaneal (TTC) arthrodesis.

5. The talus prosthesis of claim 1, wherein the anatomically contoured shape replicates the natural geometry of the asymmetrical native talus, which is divided into three main regions: a roughly cuboidal body with a dome-shaped surface that articulates with the tibia (measuring about 10-60 mm in width anteriorly and posteriorly, with a curvature radius of 10-30 mm depending on the individual), an obliquely oriented neck that angles medially at 10-30 degrees relative to the long axis of the body and slopes downward and forward, and a convex head directed anteromedially that articulates with the navicular bone, along with three inferior articular facets—posterior, middle, and anterior—facilitating articulation with the calcaneus in the complex subtalar joint.

6. The talus prosthesis of claim 1, wherein the cylindrical canal can range from 5 mm to 30 mm in diameter, depending on the size of the intramedullary nail used for tibiotalocalcaneal (TTC) arthrodesis.

7. The talus prosthesis of claim 1, wherein The prosthesis incorporates an internal gyroid lattice structure—a type of triply periodic minimal surface (TPMS)—across all configurations to support osseointegration and minimize the elastic mismatch with the host bone by featuring interconnected pores of 300 to 3000 microns that promote vascularization and bone ingrowth while maintaining mechanical strength and stability, and the cylindrical canal's surface that interfaces with the intramedullary nail has a relative density between 0.1 and 1.0 to match the stiffness between the implant and the nail.

8. A method comprising: providing an ankle prosthesis having i) Reconstruct the 3D model from the patient's CT scan, (ii) Identify and mark the area of damaged bone to be removed, (iii) Determine types of the external shape to be design, (iv) Define the lattice structure types and variables, (v) Design the graded gradient such as directional grading or biomechanically grading, (vi) Performances evaluation by simulation to analyze the stress acting on the implants, and (vii) Manufacture by 3D-printing technology;
wherein the external shape to be designed will be selected from an ellipsoidal, spherical, or anatomically conformed talus implant for use with the intramedullary nail, or as an anatomical talus implant intended to replicate the function of the original talus;
wherein talus implants are designed with two parameters: the vertical diameter and the horizontal diameter;
wherein a cylindrical volume will be subtracted from the implants to allow the intramedullary nail to pass through;
wherein anatomical talus implants can be obtained by mirroring the talus from the patient's unaffected ankle;
wherein the porous size and distribution can be determined by considering the function of the talus implants, osseointegration properties, bone contact regions, screw insertion points, and load-bearing performance.

9. The method of claim 8, wherein the vertical diameter controls the height of the implants, addressing the challenge of limb shortening.

10. The method of claim 8, wherein the horizontal diameter is considered to ensure that the implants fit securely within the tibia bone region and can accommodate the surrounding skin.

11. The method of claim 8, wherein the area in contact with the nail has a higher relative density to account for friction on the surface, while the outer surface has a lower relative density to reduce weight, as it is not subjected to forces.
